(19) United States
(12) Patent Application Publication
(10) Pub. No.: US 2016/0199539 A1
(43) Pub. Date: Jul. 14, 2016

(54) ENDOPROSTHESIS HAVING A NANOSTRUCTURED AND COATED SURFACE

(71) Applicant: AIRBUS DEFENCE AND SPACE GMBH, Ottobrunn (DE)

(72) Inventors: Ante Kurtovic, Paderborn (DE); Jürgen Steinwandel, Uhldingen-Mühlhofen (DE); Tobias Mertens, München (DE)

(73) Assignee: Airbus Defense and Space GMBH, Ottobrunn (DE)

(21) Appl. No.: 14/913,098

(22) PCT Filed: Aug. 20, 2014
(86) PCT No.: PCT/DE2014/000415

§ 371 Date: Feb. 19, 2016

(30) Foreign Application Priority Data
Aug. 22, 2013 (DE) 10 2013 013 818.3

(51) Int. Cl. A61L 27/32; A61L 27/34; A61L 27/54; A61L 27/06

(52) U.S. Cl. CPC A61L 27/32; A61L 27/06; A61L 27/34; A61L 27/54; A61L 2400/12; A61L 2300/42; A61L 2300/412; A61L 2420/02

(57) ABSTRACT

An endoprosthesis has a surface area which comprises a metal, a metal alloy, a ceramic, a platic material or a fiber-reinforced plastic material, and is uninterruptedly covered with a tissue-inductive or anticoagulant coating, at least partially. The surface area to which the coating is applied has been provided prior to that application with nanostructures having a height and a width in the range of 5 to 999 nm. A method for producing the endoprosthesis provides an endoprosthesis having these features.

### FIELD OF THE INVENTION

The invention relates to an endoprosthesis having a nanostructured surface area consisting of a metal, a metal alloy, a ceramic, a plastic material, or a fiber-reinforced plastic material, which surface area is uninterruptedly covered with a tissue-inductive or an anticoagulant coating, at least partially. The invention also relates to a method for the production of this endoprosthesis.

### BACKGROUND OF THE INVENTION

Endoprostheses covered with tissue-inductive coatings, e.g. with an osteoinductive coating consisting of a bone substitute such as hydroxylapatite, are known. The bone substitute enables good ongrowth of the surrounding tissue at the endoprosthesis. In hip prostheses, for example, good retention of the prosthesis in the bone is guaranteed even without the use of bone cement.

On the other hand, problems frequently occur concerning the adhesion of the tissue-inductive coating to the endoprosthesis.

The invention aims at improving the adhesion of a tissue-inductive or also anticoagulant coating to an endoprosthesis.

### BRIEF DESCRIPTION OF THE INVENTION

The invention relates to an endoprosthesis having a surface area consisting of a metal, a metal alloy, a ceramic, a plastic material, or a fiber-reinforced plastic material, which surface area is uninterruptedly covered with a tissue-inductive or anticoagulant coating, at least partially, wherein the surface area to which the coating is applied has been provided prior to that application with nanostructures having a height and a width in the range of 5 to 999 nm.

The invention further relates to a method for the production of an endoprosthesis having a surface area consisting of a metal, a metal alloy, a ceramic, a plastic material, or a fiber-reinforced plastic material, in which method nanostructures having a height and a width in the range of 5 to 999 nm are produced at least on parts of the surface area using a pulsed laser beam or an electron, ion or neutral particle beam, and after the production of said nanostructures a gapless tissue-inductive or anticoagulant coating is applied.

### DETAILED DESCRIPTION OF THE INVENTION

Endoprostheses are implants, which in most cases permanently remain in the body, although there are cases in which the endoprosthesis will be resorbed biologically in the course of time.

Endoprostheses particularly include bone replacement endoprostheses such as hip, knee joint, shoulder, elbow, ankle joint, and finger joint prostheses, vascular prostheses, lumen prostheses such as stents and cardiac valves.

The materials of these endoprosthesis usually comprise metal (mostly as metal alloy), ceramics, plastics and/or fiber-reinforced plastics, depending on the material requirements.

For example, the prosthesis shank of a hip prosthesis frequently comprises a titanium alloy such as TiAl6V4 or TiAl6Nb7, especially where an osteoinductive coating such as hdroxylapatite is applied which does not require the use of bone cement for fixing, a CoCrMo alloy, especially where bone cement is used for fixing, or occasionally fiber-reinforced plastics and in recent times also ceramics. Plastics are used for example in finger joint and acetabulum replacement. Stents usually comprise metal that may be a magnesium alloy, if the stent shall be resorbed biologically. Vascular prostheses usually comprise plastics, e.g. polytetrafluorethylene (PTFE), as a base material. Cardiac valves on the other hand usually comprise plastics and metal.

There are known numerous coatings that favor the ongrowth and ingrowth of tissue at and into the endoprosthesis. Such coatings are herein referred to as “tissue-inductive coatings” and as “osteoinductive coatings” in the specific case of bone tissue. Some examples of tissue-inductive coatings, which are all also osteoinductive, are hydroxylapatite, β-tricalcium phosphate and combinations thereof, tetra-calcium phosphate, kaliumhydroxyapatite, calcium sulfate, silicon apatite, magnesium apatite, bioactive glass comprising silicon, calcium, sodium, phosphorous and oxygen, collagen, collagen in combination with hydroxylapatite, growth factors, and osteoblast-binding peptide. Albumin and gelatin are examples of tissue-inductive coatings that are particularly suitable for good ongrowth and ingrowth of soft tissue. Heparin and EDTA coatings have an anticoagulant effect. According to the invention, these coating materials may contain additional pharmaceutically effective components such as antibiotics.

According to the invention, that part of the surface of an endoprosthesis that is coated with a tissue-inducing or anticoagulating coating, is provided prior to that coating with nanostructures having a height and a width in the range of 5 to 999 nm. Preferably, these nanostructures cover at least 80%, preferably at least 90% and even more preferably at least 95% of the surface of the aforementioned part of the endoprosthesis.

Said nanostructuring of the above-mentioned surface materials can be carried out using for instance a pulsed laser beam or electron, ion or neutral particle beams. Such methods are known to a person skilled in the art.

In a particularly preferred method, the entire surface area which comprises a metal, a metal alloy, a ceramic, a plastic material or a fiber-reinforced plastic material and on which said nanostructures are to be produced and which is accessible to laser irradiation, is scanned one or several times with a pulsed laser beam in such a manner that adjacent light spots of the laser beam abut one another or overlap each other without gaps, wherein the following conditions are met:

approx 0.07≦ε≦approx 2300

with

$$ɛ = \frac{{P}_{P}^{2} \cdot \sqrt{{P}_{m}} \cdot f \cdot α \cdot \sqrt{t} \cdot \sqrt{κ}}{{d}^{2} \cdot \sqrt{v} \cdot \sqrt{{T}_{V}} \cdot \sqrt{{c}_{P}} \cdot \sqrt{λ}} \cdot {10}^{3}$$

wherein:
Pₚ: pulse peak power of the emerging laser radiation [kW]
Pₘ: mean power of the emerging laser radiation [W]
t: pulse length of the laser pulses [ns], wherein t is about 0.1 ns to about 2000 ns
f: repetition rate of the laser pulses [kHz]
v: scanning speed at the work piece surface [mm/s]
d: diameter of the laser beam at the work piece [μm]
α: absorption of the laser radiation of the irradiated material [%] at normal conditions
λ: wave length of the laser radiation [nm], wherein λ=about 100 nm to about 11000 nm
Tv: boiling point of the material [K] at normal pressure
cₚ: specific heat capacity [J/kgK] at normal conditions
κ: specific thermal conductivity [W/mK] at normal conditions,
   wherein the atmosphere in which the method is carried out is vacuum, surrounding atmosphere, or an inert gas or gas mixture.

This enables complete nanostructuring of the respective surface in a reliable manner.

These nanostructures enable very good adhesion of the subsequently applied tissue-inducing or anticoagulant coating.

Numerous methods for applying said tissue-inducing or anticoagulant coatings are known to the person skilled in the art. Hydroxylapatite and other inorganic ionic coatings can be applied for instance by plasma spraying, ion implantation, sputtering, sol-gel coating, precipitation reaction, electrophoretic deposition, electrochemical deposition, electrospray deposition or laser deposition. Biological glass is preferably applied by sputtering, and organic compounds can be applied for example by electrophoresis or evaporation of a solution containing said compound.

It is known that microstructures, particularly structures in the range of 40-100 μm, on the surface of the endoprostheses will favor the ongrowth of tissue at the endoprosthesis. This also applies to the above-mentioned coatings. For this reason, the same are applied in a manner such as to exhibit a microstructured surface. This can be implemented for example by applying a very thin coating of the above-mentioned coating materials, e.g. at a thickness in the range of 30-40 μm, to a microstructured surface area of the endoprosthesis which is overlapped by nanostructures. Microstructures on the above-mentioned endoprosthesis surface materials can be produced for example by sand blasting or corundum or glass particle blasting or, in the case of titanium alloys, by titanium particles applied by plasma spraying.

Some of the above-mentioned techniques for applying the coating allow the production of a microstructured coating surface even without any microstructuring of the endoprosthesis surface area underlying said nanostructuring, by a suitable process management.

Particularly suitable for the ongrowth and ingrowth of bone tissue are mesostructured endoprosthesis surfaces with structures in a size range of 100 μm to 2000 μm underlying said nanostructuring. These structures include for example honeycomb, ball, network, cancellated bone, sponge or trabecular structures (tripods). Prior to the application of the tissue-inductive or anticoagulant coatings, these mesostructures are provided with a nanostructuring in the inventive manner as described above.

It is possible in the above described way to provide strongly adhering coatings to an endoprosthesis, whereby postoperative complications caused by peeling coating parts are effectively avoided.

1. An endoprosthesis has a surface area comprising a metal, a metal alloy, a ceramic, a plastic material or a fiber-reinforced plastic material, such that the surface area is uninterruptedly covered with a tissue-inductive or anticoagulant coating, at least partially, and the surface area to which the coating is applied has been provided prior to that application with nanostructures having a height and a width in the range of 5 to 999 nm.

2. The endoprosthesis according to claim 1, wherein at least 80% of the surface of the surface area to which the coating is applied are provided with said nanostructures.

3. The endoprosthesis according to claim 1, wherein said surface area comprises a metal alloy selected from TiAl6V4 or TiAl6Nb7.

4. The endoprosthesis according to claim 1, wherein the coating is selected from a material that comprises one or several components selected from hydroxylapatite, β-tricalcium phosphate, tetra-calcium phosphate, kaliumhydroxyapatite, calcium sulfate, silicon apatite, magnesium apatite, bioactive glass comprising silicon, calcium, sodium, phosphorous and oxygen, collagen, albumin, gelatin, growth factors, osteoblast-binding peptide, heparin, and EDTA.

5. The endoprosthesis according to claim 1, wherein the surface area that has been provided with said nanostructures comprises mesosurface structures in the range of about 100 to about 2000 μm or microsurface structures in the range of about 40 to about 100 μm under said nanostructures.

6. A method for producing an endoprosthesis having a surface area comprising a metal, a metal alloy, a ceramic, a plastic material or a fiber-reinforced plastic material, in which method nanostructures having a height and a width in the range of 5 to 999 nm are produced at least on parts of the surface area using a pulsed laser beam or an electron, ion oder neutral particle beam, the method comprising:
after the production of said nanostructures, applying a tissue-inductive or anticoagulant coating in a gapless manner.

7. The method according to claim 6, wherein the entire surface area which comprises a metal, a metal alloy, a ceramic, a plastic material or a fiber-reinforced plastic material and on which said nanostructures are to be produced and which is accessible to laser irradiation, is scanned one or several times using a pulsed laser beam in such a manner that adjacent light spots of the laser beam abut one another or overlap each other without gaps, wherein the following conditions are met:
approx 0.07≦ε≦approx 2300
with
$$ɛ = \frac{{P}_{P}^{2} \cdot \sqrt{{P}_{m}} \cdot f \cdot α \cdot \sqrt{t} \cdot \sqrt{κ}}{{d}^{2} \cdot \sqrt{v} \cdot \sqrt{{T}_{V}} \cdot \sqrt{{c}_{P}} \cdot \sqrt{λ}} \cdot {10}^{3}$$
wherein:
Pₚ: pulse peak power of the emerging laser radiation [kW]
Pₘ: mean power of the emerging laser radiation [W]
t: pulse length of the laser pulses [ns], wherein t is about 0.1 ns to about 2000 ns
f: repetition rate of the laser pulses [kHz]
v: scanning speed at the workpiece surface [mm/s]
d: diameter of the laser beam at the workpiece [μm]
α: absorption of the laser radiation of the irradiated material [%] at normal conditions
λ: wave length of the laser radiation [nm], wherein λ=about 100 nm to about 11000 nm
Tv: boiling point of the material [K] at normal pressure
cₚ: specific heat capacity [J/kgK] at normal conditions
κ: specific thermal conductivity [W/mK] at normal conditions,
wherein the atmosphere in which the method is carried out is vacuum, surrounding atmosphere or an inert gas or gas mixture.

8. The method according to claim 6, wherein the surface area comprises a metal alloy which is selected from TiAl6V4 or TiAl6Nb7.

9. The method according to claim 6, wherein the coating is a tissue-induced coating that is selected from a material which comprises one or more components selected from hydroxylapatite, β-tricalcium phosphate, kaliumhydroxyapatite, calcium sulfate, silicon apatite, magnesium apatite, collagen, albumin, gelatin, growth factors, osteoblast-binding peptide, heparin, and EDTA.

10. The method according to claim 6, wherein said nanostructures have been produced on a surface area which comprises mesosurface structures in the range of about 100 to about 2000 μm or microsurface structures in the range of about 40 to about 100 μm.

11. The method according to claim 6, wherein said tissue-induced coating is selected from a material which comprises hydroxylapatite and the coating is applied by plasma spraying, ion implantation, sputtering, sol-gel coating, precipitation reaction, electrophoretic deposition, electrochemical deposition, electrospray deposition or laser deposition.

12. The endoprosthesis according to claim 2, wherein said surface area comprises a metal alloy selected from TiAl6V4 or TiAl6Nb7.

13. The endoprosthesis according to claim 2, wherein the coating is selected from a material that comprises one or several components selected from hydroxylapatite, β-tricalcium phosphate, tetra-calcium phosphate, kaliumhydroxyapatite, calcium sulfate, silicon apatite, magnesium apatite, bioactive glass comprising silicon, calcium, sodium, phosphorous and oxygen, collagen, albumin, gelatin, growth factors, osteoblast-binding peptide, heparin, and EDTA.

14. The endoprosthesis according to claim 3, wherein the coating is selected from a material that comprises one or several components selected from hydroxylapatite, β-tricalcium phosphate, tetra-calcium phosphate, kaliumhydroxyapatite, calcium sulfate, silicon apatite, magnesium apatite, bioactive glass comprising silicon, calcium, sodium, phosphorous and oxygen, collagen, albumin, gelatin, growth factors, osteoblast-binding peptide, heparin, and EDTA.

15. The endoprosthesis according to claim 2, wherein the surface area that has been provided with said nanostructures comprises mesosurface structures in the range of about 100 to about 2000 μm or microsurface structures in the range of about 40 to about 100 μm under said nanostructures.

16. The method according to claim 7, wherein the surface area comprises a metal alloy which is selected from TiAl6V4 or TiAl6Nb7.

17. The method according to claim 7, wherein the coating is a tissue-induced coating that is selected from a material which comprises one or more components selected from hydroxylapatite, β-tricalcium phosphate, kaliumhydroxyapatite, calcium sulfate, silicon apatite, magnesium apatite, collagen, albumin, gelatin, growth factors, osteoblast-binding peptide, heparin, and EDTA.

18. The method according to claim 8, wherein the coating is a tissue-induced coating that is selected from a material which comprises one or more components selected from hydroxylapatite, β-tricalcium phosphate, kaliumhydroxyapatite, calcium sulfate, silicon apatite, magnesium apatite, collagen, albumin, gelatin, growth factors, osteoblast-binding peptide, heparin, and EDTA.

19. The method according to claim 7, wherein said nanostructures have been produced on a surface area which comprises mesosurface structures in the range of about 100 to about 2000 μm or microsurface structures in the range of about 40 to about 100 μm.

20. The method according to claim 7, wherein said tissue-induced coating is selected from a material which comprises hydroxylapatite and the coating is applied by plasma spraying, ion implantation, sputtering, sol-gel coating, precipitation reaction, electrophoretic deposition, electrochemical deposition, electrospray deposition or laser deposition.
